(19) United States
(12) Patent Application Publication
(10) Pub. No.: US 2012/0296415 A1
(43) Pub. Date: Nov. 22, 2012

(54) BIOABSORBABLE STENT

(75) Inventors: Yotaro Fujita, Shizuoka (JP); Makoto Onishi, Kanagawa (JP)

(73) Assignee: TERUMO KABUSHIKI KAISHA, Shibuya-ku (JP)

(21) Appl. No.: 13/563,199

(22) Filed: Jul. 31, 2012

Related U.S. Application Data
(63) Continuation of application No. PCT/JP2011/050053, filed on Jan. 5, 2011.

(30) Foreign Application Priority Data
Feb. 2, 2010 (JP) 2010-021542

(51) Int. Cl. A61F 2/82

(52) U.S. Cl. 623/1.15

(57) ABSTRACT

A bioabsorbable stent has a relatively high radial force and can be placed directly at the lesion without the possibility or reducing the possibility of occluding the lesion again after placement. The bioabsorbable stent is formed from a mixture composed of a bioabsorbable aliphatic polyester and an aromatic compound having one or more aromatic rings.

### CROSS REFERENCES TO RELATED APPLICATIONS

This application is a continuation application of International Application No. PCT/JP2011/050053 filed on Jan. 5, 2011 designating the U.S., and claims priority to Japanese Patent Application No. 2010-021542 filed in the Japanese Patent Office on Feb. 2, 2010. The entire content of each of these applications is hereby incorporated by reference.

### TECHNICAL FIELD

Disclosed is a bioabsorbable stent. For example, disclosed is a bioabsorbable stent for use by insertion and placement in the lumens of a living body such as a blood vessel, bile duct, trachea, esophagus, and urethra.

### BACKGROUND DISCUSSION

One way of coping with stenosis in lumens of a living body, such as blood vessels, for example, the coronary artery, is by the insertion and dilation of a balloon catheter in the stenosis to expand the blood vessel and keep the lumen open.

An example of the foregoing usage of a balloon catheter is illustrated below with reference to the angioplasty to be applied to ischemic heart disease. Patients of ischemic heart diseases (such as angina pectoris and myocardial infarction) are sharply increasing in number in Japan, for example, owing to its westernized eating habit. They can undergo percutaneous transluminal coronary angioplasty (PTCA) for the curing of a lesion in the coronary artery, and this surgical operation has widely spread. PTCA can be applied to a variety of cases, ranging from those in which the lesion is short and the stenosis occurs at one part in an early stage of PTCA, to those in which stenosis occurs at more than one part, involving distal eccentric calcification. PTCA is a procedure which can involve steps of fixing an introducer sheath to a small dissected part of the artery of the patient's leg or arm, inserting a hollow tube called a guide catheter into the blood vessel through the lumen of the introducer sheath, with the help of a guide wire advancing ahead of the guide catheter, placing the guide catheter at the entrance of the coronary artery and then withdrawing the guide wire, inserting another guide wire and a balloon catheter into the lumen of the guide catheter, advancing the balloon catheter to the lesion in the coronary artery of the patient under X-ray radiography, with the guide wire advancing ahead of the balloon catheter, placing the balloon catheter at the lesion, and dilating the balloon at a prescribed pressure for 30 to 60 seconds one to several times. In this way it is possible to expand that part of the blood vessel which has the lesion, thereby increasing the blood flow through the blood vessel. However, the above-mentioned PTCA can result in restenosis at a rate of about 30 to 40% as a result of the catheter damaging the wall of the blood vessel, thereby causing the proliferation of tunica intima which is the curing reaction in the wall of the blood vessel.

One way to prevent restenosis is to use medical devices such as a stent and atheroma excision catheter. This can be successful to some extent. The stent can include a tubular medical device to cure diseases caused by stenosis or occlusion in the blood vessel or other lumens. It can be so designed as to expand the part of stenosis or occlusion and to be placed there to ensure the lumen. The stent can be mostly made of metallic or polymeric material. It can be available in various forms, such as a tube of metallic or polymeric material with small pores formed therein and a cylinder braided with wires of metallic material or filaments of polymeric material. The placement of the stent in the blood vessel is intended to prevent or reduce the occurrence of restenosis after PTCA. In fact, however, the placement of the stent by itself is unable to prevent restenosis.

A stent loading a physiologically active agent can be used, such as immunosuppressive agent and anticancer agent. This stent can be designed to release the physiologically active agent over a prolonged period of time at that part of the lumen where the stent is placed, thereby decreasing the possibility of restenosis. An example of such stents is disclosed in EP 0 623 354 A1. It is a stent of tantalum which is coated with a mixture of a substance for curing and a biodegradable polymeric material. Another example is disclosed in Japanese Patent Laid-open No. Hei 9-56807. It is a stent of stainless steel which has thereon a drug layer and a biodegradable polymer layer for eluting drug which are formed one over the other.

In the stents disclosed in EP 0 623 354 A1 or Japanese Patent Laid-open No. Hei 9-56807, the stent body is made of metallic material such as stainless steel or tantalum and hence it can remain in the living body semipermanently after its placement. This means that the stent body can give a mechanical stress to the wall of the blood vessel, thereby causing chronic inflammation after the decomposition of the biodegradable polymer and the release of the physiologically active agent in the living body. The foregoing is applicable not only to the stents disclosed in EP 0 623 354 A1 or in Japanese Patent Laid-open No. Hei 9-56807 but also to any stent made of metallic material.

In addition, it is reported in Circulation 2002, 2649-2651 that as a result of the polymeric layer remaining semipermanently in the living body, it can bring about chronic inflammation and the deterioration of the polymeric layer can induce restenosis and intercurrent thrombosis.

Disclosed in EP 0 528 039 A1 is technology for forming a stent body with polylactic acid. In EP 0 528 039 A1, the polylactic acid constituting the stent body decomposes and the stent body disappears. Consequently, there can be no possibility of the chronic inflammation occurring as a result of the stent giving mechanical stress to the wall of the blood vessel after placement in the living body for a long period of time. Thus, the stent mentioned above can be less or not very invasive to the patient.

### SUMMARY

The above-mentioned stent made of polylactic acid, as disclosed in EP 0 528 039 A1, can still have a problem of being poor in radial force because it is made of polylactic acid lacking mechanical strength. With a poor radial force, for example, it may not be possible to place the stent at the desired position (lesion). The stent can shrink (recoil) inward after placement at the lesion, thereby occluding the lesion again.

Disclosed is a bioabsorbable stent that has a high radial force and hence can be placed at the lesion, for example, without the possibility or with a reduced possibility of occluding the lesion again after placement.

According to an exemplary aspect, a stent formed from a bioabsorbable aliphatic polyester and an aromatic compound having one or more aromatic rings can exhibit a high radial force which permits it to be placed at the desired position (lesion) in the living body, without the possibility or with a reduced possibility of occluding the lesion again after placement.

According to an exemplary aspect, disclosed is a bioabsorbable stent formed from a mixture composed of a bioabsorbable aliphatic polyester and an aromatic compound having one or more aromatic rings.

The bioabsorbable stent according to an exemplary aspect has a high radial force and hence it can be placed at the lesion without the possibility or with a reduced possibility of occluding the lesion again after placement at the lesion.

According to an exemplary aspect, disclosed is a method of forming a bioabsorbable stent, the method comprising forming the bioabsorbable stent by subjecting the mixture comprising the bioabsorbable aliphatic polyester and the aromatic compound having one or more aromatic rings to blow molding.

### BRIEF DESCRIPTION OF THE DRAWINGS

FIG. 1 is a side view of a stent according to an exemplary embodiment. In FIG. 1, 1 denotes the stent, C denotes a linear member, D denotes a roughly rhombic element, E denotes an annular unit, and F denotes a connecting member.

FIG. 2 is an enlarged cross sectional view taken along the line A-A in FIG. 1, according to an exemplary embodiment. In FIG. 2, 1 denotes the stent, 2 denotes the stent body, 3 denotes a physiologically active agent layer, and 4 denotes a biodegradable polymer layer.

FIG. 3 is an enlarged longitudinal sectional view taken along the line B-B in FIG. 1, according to an exemplary embodiment. In FIG. 3, 1 denotes the stent, 2 denotes the stent body, 3 denotes a physiologically active agent layer, and 4 denotes a biodegradable polymer layer.

FIG. 4 is another enlarged cross sectional view taken along the line A-A in FIG. 1, according to an exemplary embodiment. In FIG. 4, 1 denotes the stent, 2 denotes the stent body, 5 denotes a biodegradable polymer layer, and 6 denotes a physiologically active agent layer.

FIG. 5 is another enlarged longitudinal sectional view taken along the line B-B in FIG. 1, according to an exemplary embodiment. In FIG. 5, 1 denotes the stent, 2 denotes the stent body, 5 denotes a biodegradable polymer, and 6 denotes a physiologically active agent.

FIG. 6 is another enlarged cross sectional view taken along the line A-A in FIG. 1, according to an exemplary embodiment. In FIG. 6, 1 denotes the stent, 2 denotes the stent body, and 6 denotes a physiologically active agent.

FIG. 7 is another enlarged cross sectional view taken along the line A-A in FIG. 1, according to an exemplary embodiment. In FIG. 7, 1 denotes the stent, 2 denotes the stent body, 6 denotes a physiologically active agent, and 7 denotes a bioabsorbable aliphatic polyester.

FIG. 8 is a side view of a stent according to an exemplary embodiment.

### DETAILED DESCRIPTION

According to an exemplary embodiment, provided is a bioabsorbable stent (also referred to as stent hereinafter) which is formed from a mixture composed of a bioabsorbable aliphatic polyester and an aromatic compound having one or more aromatic rings. The stent can contain an aromatic compound having one or more aromatic rings. This aromatic compound can cause the molecular chains of the aliphatic polyester to arrange themselves regularly on account of the stacking action of the aromatic rings, with the result that the stent can increase in mechanical strength (radial force) while possessing adequate flexibility. The stent can be placed directly at the lesion and can remain there without shrinking (recoiling) inward to prevent occluding of the lesion again. In addition, the stent can contain a bioabsorbable aliphatic polyester, which undergoes chemical decomposition, so that the stent can be eventually biodegraded and absorbed into the living body. Thus, the stent can reduce or eliminate the possibility of causing chronic inflammation due to its mechanical stress given to the wall of the blood vessel after placement in the living body for a long period of time. For example, the stent can be non-invasive or not very invasive to the living body. The term “radial force” used in this specification includes the force (rebound) in a radial direction of the blood vessel which the stent exerts on the wall of the blood vessel. It denotes a value which is obtained when a stent, measuring 3 mm in outside diameter and 10 mm long, is compressed by 1 mm at a compression rate of 10 mm/min and its radial force (rebound) is measured by using an autograph (Model AG-IS made by Shimadzu Corporation).

An exemplary stent contains a bioabsorbable aliphatic polyester, which undergoes decomposition and absorption in a living body with time. For example, in an exemplary embodiment, it does not stay in a living body for a long period of time and it does not give any mechanical stress to the wall of the blood vessel, which reduces or eliminates the possibility of causing chronic inflammation. For example, it is non-invasive or not very invasive to a living body. It may contain physiologically active agents (as described later), which can be gradually released with time as the bioabsorbable aliphatic polyester undergoes biodegradation and absorption.

The bioabsorbable aliphatic polyester mentioned above is not specifically restricted, but it can be one which is highly stable in the living body. Examples thereof include the following: polylactic acid, polyglycolic acid, copolymer of lactic acid and glycolic acid, polycaprolactone, copolymer of lactic acid and caprolactone, copolymer of glycolic acid and caprolactone, polytrimethylene carbonate, copolymer of lactic acid and trimethylene carbonate, copolymer of glycolic acid and trimethylene carbonate, polydioxane, polyethylene succinate, polybutylene succinate, polybutylene succinate-adipate, polyhydroxybutylic acid, and polymalic acid. Exemplary among them are polylactic acid, polyglycolic acid, copolymer of lactic acid and glycolic acid, polycaprolactone, copolymer of lactic acid and caprolactone, copolymer of glycolic acid and caprolactone, polytrimethylene carbonate, copolymer of lactic acid and trimethylene carbonate, copolymer of glycolic acid and trimethylene carbonate, polydioxane, polyethylene succinate, polybutylene succinate, and polybutylene succinate-adipate. Exemplary among them are polylactic acid, polyglycolic acid, copolymer of lactic acid and glycolic acid, copolymer of lactic acid and trimethylene carbonate, and copolymer of glycolic acid and trimethylene carbonate. They can be degradable in a living body and yet they can exhibit high medical safety. The above-mentioned bioabsorbable aliphatic polyesters may be used alone or in combination with one another as a mixture. In addition, the aliphatic ester as a constituent of the bioabsorbable aliphatic polyester may contain lactic acid of any optical isomer. The polylactic acid may include L-polylactic acid, D-polylactic acid, and D,L-polylactic acid. The bioabsorbable aliphatic polyester in copolymer form is not specifically restricted in structure. It may be in the form of block copolymer, random copolymer, graft copolymer, or alternating copolymer. Further, the bioabsorbable aliphatic polyester may be obtained commercially or by synthesis. Any suitable method can be used for synthesis. For example, polylactic acid may be obtained from L-lactic acid or D-lactic acid, whichever is desired, by dehydration and condensation through the lactide method or the direct polymerization method.

The bioabsorbable aliphatic polyester mentioned above is not specifically restricted in weight-average molecular weight. It can be absorbable in a living body. The weight-average molecular weight can be 10,000 to 3,000,000, for example, 20,000 to 2,000,000, for example, 50,000 to 1,000,000, for example, 60,000 to 500,000, for example, 80,000 to 300,000. With the foregoing weight-average molecular weight, the bioabsorbable aliphatic polyester can exhibit satisfactory biodegradability, bioabsorbability, moldability, and mechanical strength. The “weight-average molecular weight” may be determined by any suitable method, such as GPC, light scattering method, viscosity measurement method, and mass spectrometry (such as TOFMASS). In this specification, the “weight-average molecular weight” denotes the value determined by using polystyrene, whose molecular weight is known by GPC, as the reference material.

An exemplary stent also contains an aromatic compound having one or more aromatic rings. The aromatic rings existing in the aromatic compound can cause the molecular chains of the aliphatic polyester to regularly arrange by its stacking action. This can lead to improvement in the stent's mechanical strength (radial force). Having adequate flexibility, the stent can be placed directly at the lesion. For example, once it is placed at the lesion, an exemplary stent does not shrink (or recoil) inward to prevent occluding of the lesion again. The aromatic compound can become stable and can crystallize easily as its aromatic rings (such as, for example, benzene rings) come close together. For example, the stent can exhibit flexibility as well as high radial force (or high mechanical strength), so that it can be placed directly at the lesion. For example, an exemplary stent which has been placed at the lesion does not recoil.

The aromatic compound mentioned above is not restricted in structure. It can have one or more aromatic rings. It can have a hydroxyl or carboxyl group, which can form a chemical linkage such as chemical bonding with the reactive functional group (for example, hydroxyl or carboxyl group) in the bioabsorbable aliphatic polyester. The aromatic compound having a hydroxyl or carboxyl group can contribute to the stent's mechanical strength and can permit the stent to be placed directly at the lesion, without recoiling after placement.

Examples of the aromatic compound include the following: 2-hydroxybenzoic acid (salicylic acid), 3-hydroxybenzoic acid, 4-hydroxybenzoic acid, 2,3-dihydroxybenzoic acid, 2,4-dihydroxybenzoic acid, 2,5-dihydroxybenzoic acid, 2,6-dihydroxybenzoic acid, 3,5-dihydroxybenzoic acid, 2-hydroxycinnamic acid, 3-hydroxycinnamic acid, 4-hydroxycinnamic acid, 4-hydroxy-2-methoxycinnamic acid, 4-hydroxy-3-methoxycinnamic acid, 3,4-dihydroxycinnamic acid, mandelic acid, and tyrosine. The foregoing aromatic compound may be an iodized one, which permits the stent to be visible under X-ray radiography. This can enable the operator to easily confirm the stent's position with the help of X-rays. The iodized aromatic compound is not specifically restricted. It may be commercially available or may be synthesized by any suitable method. Exemplary among the foregoing aromatic compounds are 2-hydroxybenzoic acid, 3-hydroxybenzoic acid, 4-hydroxybenzoic acid, 2-hydroxycinnamic acid, 3-hydroxycinnamic acid, 4-hydroxycinnamic acid, mandelic acid, and tyrosine, and iodides thereof. Exemplary among them are 2-hydroxybenzoic acid, 3-hydroxybenzoic acid, 4-hydroxybenzoic acid, 4-hydroxycinnamic acid, mandelic acid, and tyrosine, and iodides thereof. The foregoing aromatic compounds may be used alone or in combination with one another as a mixture.

An exemplary stent can be made of the bioabsorbable aliphatic polyester and the aromatic compound both mentioned above. The two components may be mixed in any ratio without specific restrictions, for example, such that the resulting stent exhibits excellent biodegradability and bioabsorbability, a high radial force (mechanical strength), adequate flexibility, and low invasiveness. An exemplary mixing ratio of the bioabsorbable aliphatic polyester to the aromatic compound (bioabsorbable aliphatic polyester:aromatic compound by mass) ranges from 100:0.1 to 100:9, for example, from 100:0.5 to 100:8, for example, from 100:1 to 100:7. Such mixing ratios can be desirable for the stent to exhibit excellent biodegradability and bioabsorbability, a high radial force (mechanical strength), adequate flexibility, and low invasiveness.

An exemplary stent is not specifically restricted in shape and can be strong enough to be stably placed in the lumen of a living body. An exemplary shape is a cylinder braided with filaments or a tube having pores in its wall. In addition, an exemplary stent may be either of balloon-expandable type or self-expandable type. It can have an adequate size in conformity with the position where it is placed. The outside diameter of the stent before expansion can be 1.0 to 5.0 mm, for example, 1.50 to 4.50 mm. The length can be 5 to 100 mm, for example, 7 to 50 mm. The stent can have an adequate wall thickness which is not specifically restricted. The wall thickness can permit the stent to be placed at the lesion and exhibit a radial force suitable for keeping the lesion open after placement and ensuring blood flow. The wall thickness can be 1 to 1000 μm, for example, 50 to 300 μm.

An exemplary stent can contain a mixture of the bioabsorbable aliphatic polyester and the aromatic compound both mentioned above. The stent may be formed only from a mixture of the bioabsorbable aliphatic polyester and the aromatic compound or from said mixture incorporated with additional components. Such additional components are not specifically restricted. Any ones suitable for use for stents are acceptable. They can include physiologically active agents and biodegradable polymers.

The physiologically active agents are not specifically restricted and can be selected as desired. The physiologically active agents can produce the effect of reducing or preventing restenosis and occlusion after the stent has been placed at the lesion in the lumen. Examples include the following: anticancer drug, immunosuppresive drug, antibiotic, antirheumatic drug, antithrombotic drug, HMG-CoA (hydroxymethylglutaryl CoA) reductase inhibitor, ACE inhibitor (andiotensin conversion enzyme inhibitor), calcium antagonist, antihyperlipidemic drug, integrin inhibitor, antiallergic drug, antioxidant, GPIIb/IIIa antagonist, retinoid, flavonoid, carotenoid, lipid improver, DNA synthesis inhibitor, tyrosine kinase inhibitor, antiplatelet drug, drug to prevent proliferation of smooth muscle of blood vessel, antiinflammatory drug, tissue-derived biomaterial, interferon, and NO generation promoting agent.

The anticancer drug can include, for example, vincristine, vinblastine, vindesine, irinotecan, pirarubicin, paclitaxel, docetaxel, and methotrexate.

The immunosuppresive drug can include, for example, sirolimus, everolimus, biolimus, tacrolimus, azathioprine, ciclosporin, cyclophosphamide, mycophenolate mofetil, gusperimus, and mizoribine.

The antibiotic can include, for example, mitomycin, adriamycin, doxorubicin, actinomycin, daunorubicin, idarubicin, pirarubicin, aclarubicin, epirubicin, peplomycin, and zinostatin stimalamer.

The antirheumatic drug can include, for example, methotrexate, sodium thiomalate, penicillamine, and lobenzarit.

The antithrombotic drug can include, for example, heparin, aspirin, antithrombin drug, ticlopidine, and hirudin.

The HMG-CoA reductase inhibitor can include, for example, cerivastatin, cerivastatin sodium, atorvastatin, rosuvastatin, pitavastatin, fluvastatin, fluvastatin sodium, simvastatin, lovastatin, and pravastatin.

The ACE inhibitor can include, for example, quinapril, perindopril erbumine, trandolapril, cilazapril, temocapril, delapril, enalapril maleate, lisinopril, and captopril.

The calcium antagonist can include, for example, hifedipine, nilvadipine, diltiazem, benidipine, and nisoldipine.

The antihyperlipidemic drug can include, for example, probucol. The integrin inhibitor can include, for example, AJM300. The antiallergic drug can include, for example, tranilast. The antioxidant can include, for example, catechins, anthocyanin, proanthocyanidin, lycopene, and β-carotene. Exemplary among cathechins is epigallocathechin gallate. The GPIIb/IIIa antagonist can include, for example, abciximab.

The retinoid can include, for example, all-trans retinoic acid. The flavonoid can include, for example, epigallocatechin, anthocyanin, and proanthocyanidin. The carotenoid can include, for example, β-carotene and lycopene. The lipid improver can include, for example, eicosapentaenoic acid.

The DNA synthesis inhibitor can include, for example, 5-FU. The tyrosine kinase inhibitor can include, for example, genistein, tyrphostine, and erbstatin. The antiplatelet drug can include, for example, ticlopidine, cilostazol, and clopidogrel. The antiinflammatory drug can include, for example, such steroid as dexamethasone and prednisolone.

The tissue-derived biomaterial can include, for example, EGF (epidermal growth factor), VEGF (vascular endothelial growth factor), HGF (hepatocyte growth factor), PDGF (platelet derived growth factor), and BFGF (basic fibrolast growth factor).

The interferon can include, for example, interferon-γ1a. The NO generation promoting substance can include, for example, L-arginine.

The foregoing physiologically active agents may be used alone or in combination with one another as a mixture. At least one of them can be contained in the bioabsorbable stent for positive prevention of restenosis. For example, whether one or two or more of the physiologically active agents are used can depend on the specific case. The physiologically active agents to be contained in the bioabsorbable stent are not restricted in amount and can be selected depending on the specific case. An exemplary amount is 1 to 50 wt %, for example, 5 to 20 wt %, for the total amount of the bioabsorbable aliphatic polyester and the aromatic compound both mentioned above. This amount can be sufficient for positive prevention of restenosis and occulusion.

The biodegradable polymer is a polymer which can gradually decompose after the stent has been placed at the lesion. It is not specifically restricted. For example, it can have no adverse effect on the living body of human or animal. Those excelling in living body stability are exemplary. Examples of the biodegradable polymer include not only the above-mentioned bioabsorbable aliphatic polyester but also at least one polymer selected from poly-α-amino acid, collagen, laminin, heparan sulfate, fibronectin, vitronectin, chondroitin sulfate, hyaluronic acid, and polymer of cinnamic acid or cinnamic acid derivative, and copolymer of the constituent of said polymer with an arbitrary monomer and a mixture of said polymer and copolymer. The term “mixture” used in this specification is a broad concept covering complexes such as polymer alloys. In addition, the biodegradable polymer is not specifically restricted in the weight-average molecular weight, which may range from 10,000 to 1,000,000, for example, from 20,000 to 500,000, for example, from 50,000 to 200,000. The “weight-average molecular weight” just mentioned above may be measured by any suitable method including GPC, light-scattering method, viscosity method, and mass spectrometry (such as TOFMASS). The term “weight-average molecular weight” used in this specification denotes the value determined by GPC that employs polystyrene of known molecular weight as the reference substance. The above-mentioned biodegradable polymers may be used alone or in combination with one another as a mixture.

The foregoing biodegradable polymer may be contained, for example, when a certain degree of biodegradability of the bioabsorbable stent is desired. The content of the biodegradable polymer in the bioabsorbable stent is not specifically restricted. It can be properly established according to the patient's graveness, case, and past history in addition to the certain degree of biodegradability. The content of the biodegradable polymer can be 1 to 50 mass %, for example, 5 to 20 mass %, for the total amount of the bioabsorbable aliphatic polyester and aromatic compound.

An exemplary stent can be produced by any suitable methods, alone or in combination, without specific restrictions. Examples of such suitable methods include blow molding, extrusion molding, injection molding, rotational molding, blow molding, transfer molding, press molding, and solution casting. Exemplary among these methods are extrusion molding, blow molding, and injection molding, with blow molding being exemplary.

The stent can have sufficient strength in its radial direction (or radial force) so that it withstands compressive force in its radial direction or the structural load which it receives when it supports the wall of the lumen such as blood vessel. The strength in radial direction is associated with the strength in circumferential direction (radial force) of the stent. The stent can have the higher strength in circumferential direction. In addition, the stent can have sufficient flexibility and strength in its lengthwise direction so that it withstands expansion and bending which it experiences during operation and after placement. For example, the stent can have strength as well as flexibility in its lengthwise direction.

Blow molding among the foregoing molding methods can be designed to apply a stress to a mixture of the bioabsorbable aliphatic polyester and the aromatic compound, thereby deforming the mixture in the circumferential (or radial) direction. The applied stress can bring about the molecular orientation of the bioabsorbable aliphatic polyester in the direction of stress application, thereby imparting improved mechanical characteristics (radial force) in the circumferential direction (or radial direction). The “molecular orientation” means the relative orientation of the polymer chains along the longitudinal or circumferential (radial) direction of the polymer chains. The “molecular orientation” is intended for the relative orientation of the polymer chains along the circumference (or radius). Blow molding can cause the bioabsorbable aliphatic polyester constituting the stent to undergo molecular orientation in the circumferential (radial) direction, thereby making the resulting stent improve in mechanical strength (radial force). The stent can be formed by blow molding so that the resulting stent can be placed directly at the lesion. Additionally, mechanical failure and shrinking (or recoiling) inward after placement can be prevented or reduced.

As mentioned above, an exemplary stent can be formed by blow molding from a mixture of a bioabsorbable aliphatic polyester and an aromatic compound having one or more aromatic rings. The following is a detailed description of an exemplary method for producing an exemplary stent.

For example, the first step of the production method is to form a parison by a suitable method such as extrusion molding from a mixture of a bioabsorbable aliphatic polyester and an aromatic compound. The resulting parison is not specifically restricted in size. For example, the outside diameter of the parison can be 0.9 to 2.0 mm, for example, 1.00 to 1.80 mm in the case of blow molding to the outside diameter of 3.00 mm. The length of the parison can be 5 to 100 mm, for example, 7 to 50 mm. The wall thickness of the parison can be 100 to 1,000 μm, for example, 200 to 600 μm.

In the next step, for example, the parison can be expanded by introduction of a fluid thereinto under pressure, thereby deforming the parison in its radial direction. In this way the blow molding can be completed to form the stent. The foregoing process may be carried out in such a way that the parison is stretched with one end, with the other end thereof fixed, in its axial direction, or the parison is stretched with both ends. Alternatively, the axial stretching may be accomplished before, during, or after (or during and after) expansion in the radial direction.

The blow molding may have an initial step of positioning the parison in a cylindrical member or mold. The mold restricts the deformation of the parison's outside diameter or surface in conformity with the inside diameter of the mold so that the deformation of the parison in its radial direction is controlled. The inside diameter of the mold may be smaller than the desirable diameter of the parison. The deformation of the parison in its radial direction may be controlled by introduction of a fluid at a properly controlled temperature and pressure in place of the mold or in combination with the mold.

The blow molding may be accomplished under any conditions so long as it produces the stent of desired shape. For example, the blow molding temperature can be 40 to 250° C., for example, 50 to 180° C. The pressure of the fluid to be introduced into the parison during blow molding can be 0.01 to 10.0 MPa, for example, 0.1 to 8.0 MPa. The duration of fluid introduction can be 10 to 1,200 seconds, for example, 20 to 600 seconds. Blow molding under the foregoing conditions can permit the bioabsorbable aliphatic polyester to undergo adequate molecular orientation in the circumferential direction, thereby imparting adequate mechanical strength (radial force) to the stent. The parison may be heated before, during, or after its deformation. For example, the parison may be heated by introduction of a fluid therein or thereon which is kept at the above-mentioned temperature. This fluid may be the same one as used to apply a pressure into the parison. In addition, the parison may be heated by moving it with a heating element or nozzle juxtaposed thereto. Also, the parison may be heated by the mold. In this case, the mold may be heated by heating any element on or in the mold or an element juxtaposed to the mold. The fluid can be any suitable fluid, which may be used as such without specific restrictions. Examples of such fluid include air, compressed air, dry nitrogen, oxygen, argon, etc.

The blow molding process may start with a step of closing or sealing one end of the parison. The sealed or closed end may be opened after molding. The sealed parison may be pressurized by introduction of a fluid thereinto. The fluid may be introduced in such a way as to expand the parison in the radial direction. The parison may be permanently deformed by heat-setting by keeping the pressure in it, the tension along its axis, and its temperature higher than its ambient ones. The heat-setting may be achieved by keeping the parison at an adequate temperature, for example, at the desired temperature mentioned above. The duration of time for this purpose may be about one minute to two hours, for example, about two minutes to 10 minutes, although it is not specifically restricted. After the heat-setting, the parison may be cooled and cut in a desired shape, size, and length. After cooling, the deformed parison can retain the length and shape conforming to the inside of the mold.

After the blow molding process, the parison can be formed into a desired shape, which can subsequently undergo laser etching, chemical etching, laser cutting, or the like according to the desired stent structure. In this way there can be obtained the stent. After the blow molding process, the pores may be formed by coating the surface of the parison with a perforated pattern and removing those parts except for the perforated pattern with the help of a laser beam or a chemical solution. The laser cutting may be accomplished by making pores in the surface of the tubular product with the help of a laser beam which is scanned according to a pattern information stored in a computer.

The exemplary stent, which is produced by the foregoing process, can excel in mechanical strength with adequate flexibility and high radial force. In addition, the stent, which contains a bioabsorbable aliphatic polyester, can be eventually decomposed in the living body and absorbed into the living body. Owing to its high radial force, the stent can be placed at the desired position (lesion), and it can prevent occluding of the lesion again without shrinking (recoiling) inward once it is placed at the lesion. After fulfilling its function to prevent acute vascular occlusion or restenosis as a stent, the stent can be decomposed and absorbed in the living body. This can lead to a limited possibility of the stent causing restenosis and complication of thrombosis in the late phase. As the result, the stent can reduce or prevent chronic inflammation caused by mechanical stress which a comparative stent gives to the wall of the blood vessel after its placement for a long period of time. For example, an exemplary stent is non-invasive or not very invasive to the patient.

The structure of an exemplary stent will be described below in more detail with reference to one embodiment illustrated in the accompanying drawings. The following description is not intended to restrict the structure of the stent.

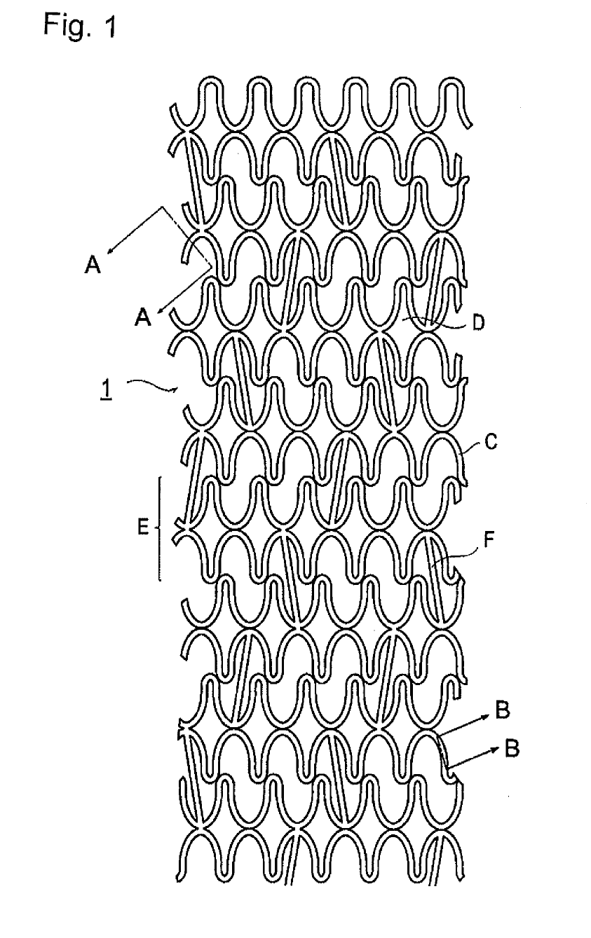

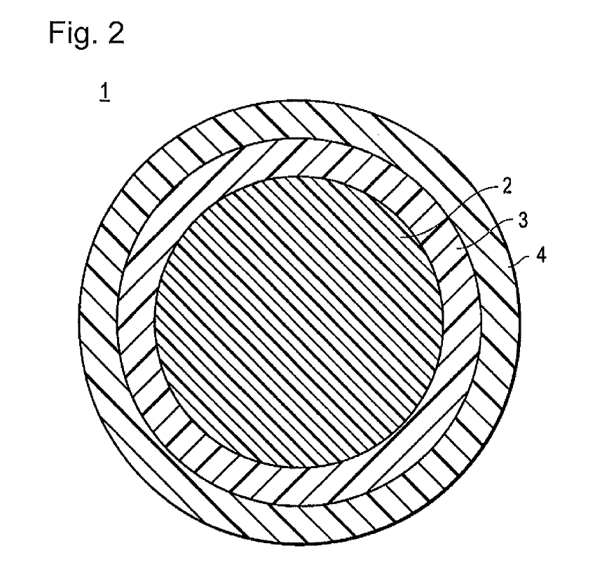

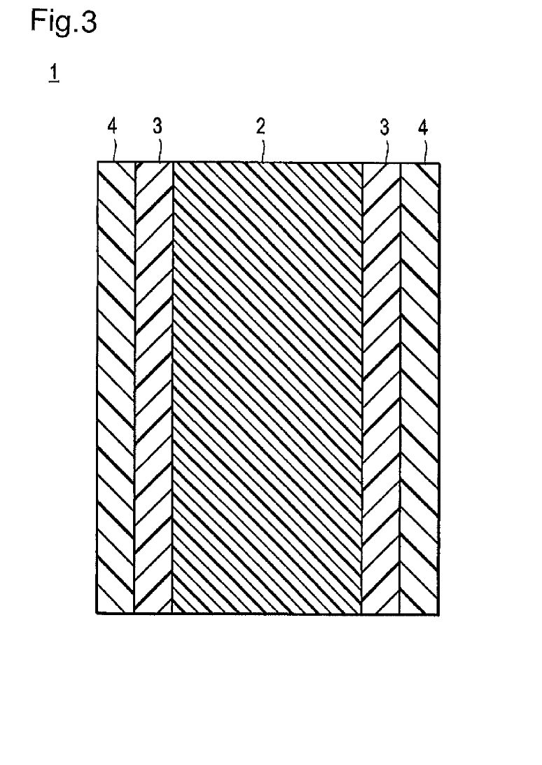

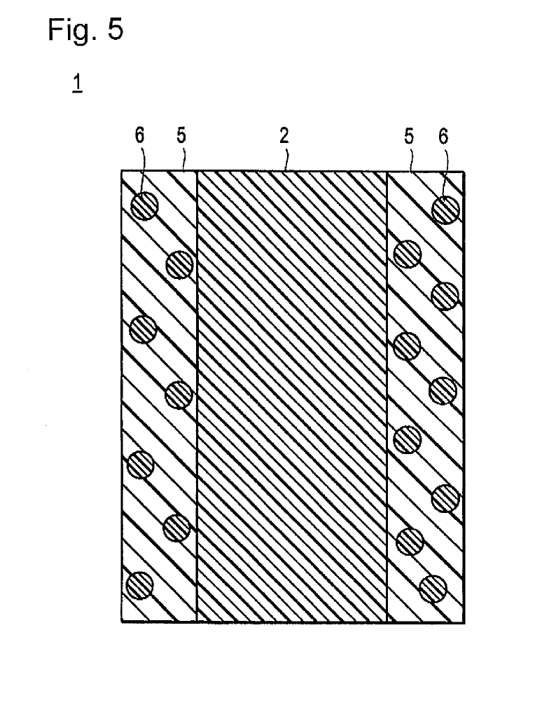

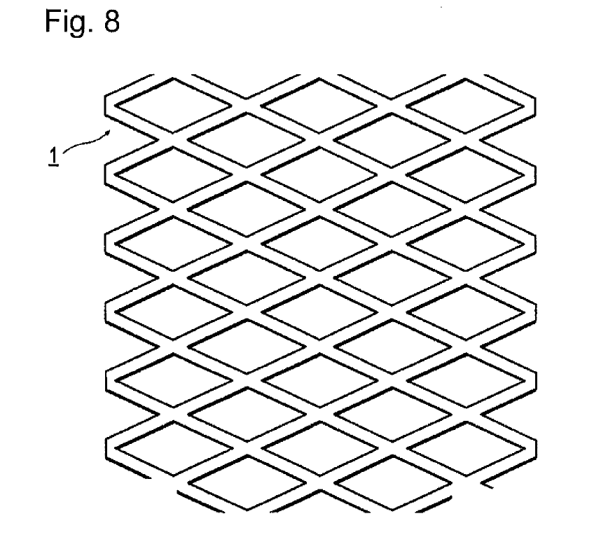

FIG. 1 is a side view of an exemplary stent. FIGS. 2, 4, 6, and 7 are enlarged cross sectional views each taken along the line A-A in FIG. 1. FIGS. 3 and 5 are enlarged longitudinal sectional views each taken along the line B-B in FIG. 1. An exemplary stent is not limited to the one shown in FIG. 1. For example, it may be the one with lattice structure as shown in FIG. 8.

The following is a detailed description of elements constituting the exemplary stent 1 shown in FIG. 1.

The stent 1 (or the stent body 2) is a cylindrical body having open ends and extending in its axial direction. The wall of the cylindrical body has a large number of cut openings passing through it. The cut openings deform so that the cylindrical body expands and shrinks in its radial direction. Thus the stent maintains its shape after it is placed in the lumen of a living body such as blood vessel and bile duct.

The stent 1 (or the stent body 2) according to the exemplary embodiment shown in FIG. 1 is a bioabsorbable stent made of a mixture composed of a bioabsorbable aliphatic polyester and an aromatic compound having one or more aromatic rings as mentioned above. It is composed of a roughly rhombic element D with a cut opening therein, as a basic unit. A plurality of roughly rhombic elements continuously arranged in the circumferential direction constitute the annular unit E. Each annular unit E is joined to its adjacent ones through linear connecting members F. In this way the annular units E in plural number are continuously arranged in the axial direction, with a portion of them joined together. The stent 1 (or the stent body 2) constructed as mentioned above constitutes a cylindrical body which has open ends and extends in its axial direction. In addition, the wall of the cylindrical body has roughly rhombic cut openings, and the cylindrical body expands and shrinks in its axial direction as the cut openings deform.

The exemplary stent is not restricted to the exemplary structure shown in FIG. 1. The stent can be a cylindrical body axially extending between open ends which has a large number of cut openings in its wall and the cylindrical body expands and shrinks in its radial direction as these cut openings deform. Therefore, the stent can have a coil-like structure. The elastic thin members constituting the stent (or stent body) may have any sectional shape, including rectangle, circle, ellipsoid, polygon, etc.

The exemplary stent 1 shown in FIG. 2 is constructed such that the stent body 2 carries thereon a layer that releases a physiologically active agent, said layer being composed of a physiologically active agent and a biodegradable polymer. In FIG. 2, the layer that releases a physiologically active agent is composed of the physiologically active agent layer 3, which is in contact with the surface of the stent body 2, and the biodegradable polymer layer 4 which entirely covers the physiologically active agent layer 3.

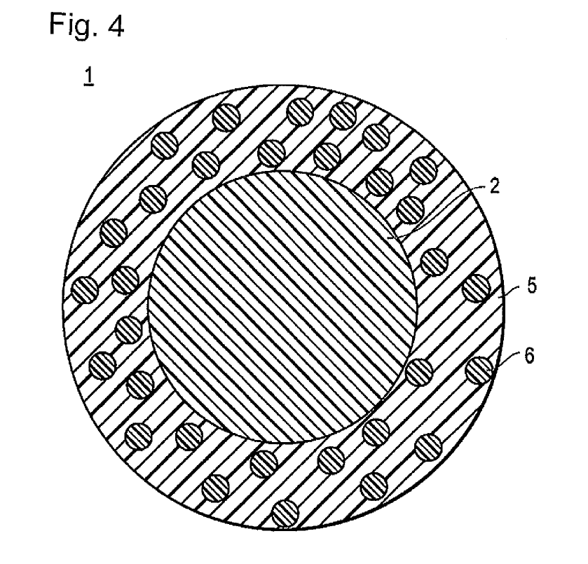

The stent 1 shown in FIG. 4 is constructed such that the stent body 2 carries thereon a layer that releases a physiologically active agent, said layer being composed of a physiologically active agent and a biodegradable polymer. The layer that releases a physiologically active agent is composed of a physiologically active agent 6 and a biodegradable polymer 5 which are mixed together.

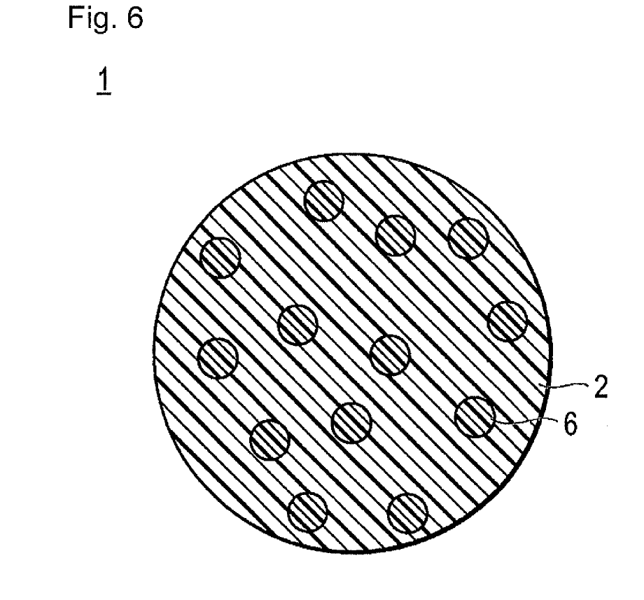

The stent 1 shown in FIG. 6 has a stent body 2 which contains a physiologically active agent 6 dispersed or embedded therein. The stent body 2 may carry thereon a layer (not shown) that releases a physiologically active agent, said layer being composed of a physiologically active agent and a biodegradable polymer.

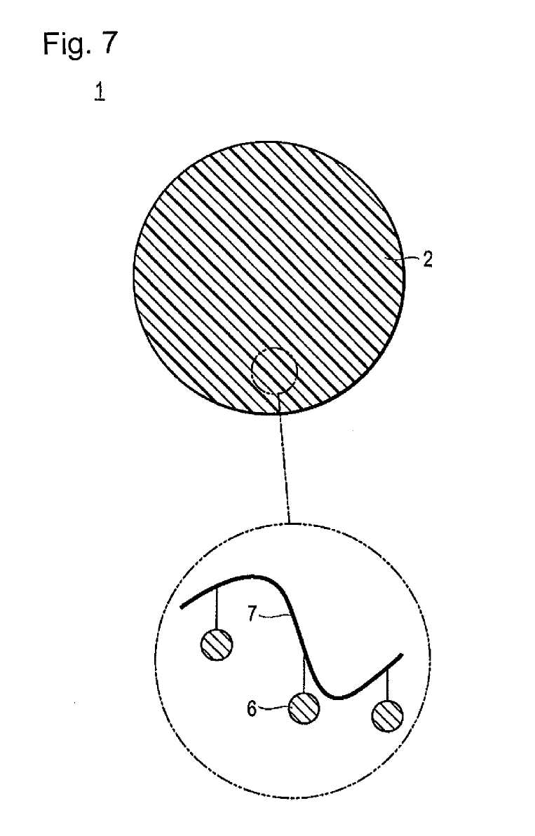

The stent 1 shown in FIG. 7 is constructed such that the stent body 2 carries a physiologically active agent 6 chemically bonding thereto. The inset in FIG. 7 is an enlarged view of the stent 1 which shows that the bioabsorbable aliphatic polyester 7 constituting the stent body 2 has a physiologically active agent 6 chemically bonding directly thereto. For example, the bioabsorbable aliphatic polyester 7 has the physiologically active agent in its side chains, which constitutes the so-called prodrug structure. The stent body 2 may additionally have a layer (not shown) which releases a physiologically active agent, said layer being composed of a physiologically active agent and a biodegradable polymer, as shown in FIGS. 2 and 4.

The stent 1 which is shown in FIG. 3, is the same one as shown in FIG. 2. FIG. 3 is a longitudinal sectional view taken along the line B-B in FIG. 1. The stent body 2 is composed of linear members C constituting the bioabsorbable stent. Each of the members C carries thereon a layer that releases a physiologically active agent (the physiologically active agent 3 layer and the biodegradable polymer layer 4). The layer that releases a physiologically active agent may cover the stent body 2 entirely or partly. The entire covering is schematically shown in FIG. 3.

The stent 1 which is shown in FIG. 5, is the same one as shown in FIG. 4. FIG. 5 is a longitudinal sectional view taken along the line B-B in FIG. 1. The stent body 2 is composed of linear members C, each of which carries thereon entirely a layer that releases a physiologically active agent (a layer formed from a mixture of a physiologically active agent 6 and a biodegradable polymer 5). The layer that releases a physiologically active agent may cover the stent body 2 entirely or partly. The entire covering of the entire surface of the linear members C formed of bioabsorbable stent constituting the stent body 2 is schematically shown in FIG. 5.

The layer that releases a physiologically active agent can be formed at least on that part of the linear member C which comes into direct contact with the tissue of a living body. This can cause the physiologically active agent released from the layer to be absorbed directly into the tissue of a living body without being dissolved in the body fluid (blood). The physiologically active agent locally administered in this way can exhibit its physiological action more effectively.

In the case where the stent body 2 contains a physiologically active agent or the stent 1 has a physiologically active agent layer or a layer that releases a physiologically active agent, the stent 1 can release the physiologically active agent after it has been placed at the lesion in a living body, thereby reducing or preventing restenosis. The bioabsorbable aliphatic polyester and the biodegradable polymer can be completely decomposed in the living body.

It is optional for the stent body 2 to have the physiologically active agent layer 3 or the biodegradable polymer layer 4 as mentioned above. The stent body 2 may have thereon the physiologically active agent layer 3 or the biodegradable polymer layer 4. In the latter case, the physiologically active agent layer 3 can have a thickness not harmful to the performance of the stent body 2, such as easy delivery to the lesion and low stimulus to the blood vessel. A thickness can be 1 to 100 μm, for example, 1 to 50 μm, for example, 1 to 20 μm, so that the physiologically active agent fully produces its effect. Likewise, the biodegradable polymer layer 4 can have an adequate thickness not harmful to the performance of the stent body 2, such as easy delivery to the lesion and low stimulus to the blood vessel as with the physiologically active agent layer 3. A thickness can be 1 to 75 μm, for example, 1 to 25 μm, for example, 1 to 10 μm. The physiologically active agent and biodegradable polymer that can be used are not specifically restricted, but any suitable ones, such as mentioned above, may be employed.

Any suitable method may be employed without specific restrictions to form the physiologically active agent layer 3 on the surface of the stent body 2. It is exemplified below. A method can include melting a physiologically active agent and applying the resulting melt onto the surface of the stent body 2. A method can include dissolving a physiologically active agent in a solvent and dipping the stent body 2 in the resulting solution, followed by solvent removal by evaporation or the like. A method can include spraying the foregoing solution onto the stent body 2, followed by solvent removal by evaporation or the like.

For example, the dipping and spraying methods which employ a solution dissolving only a physiologically active agent in a solvent are simple and exemplary in the case where the physiologically active agent can be dissolved in a solvent which makes the surface of the stent body 2 highly wettable.

The physiologically active agent layer 3 can be coated with the biodegradable polymer layer 4. More than one kind of biodegradable polymer may be used in the polymer layer.

Any suitable method may be employed without specific restrictions to form the biodegradable polymer layer 4. It is exemplified below. A method can include melting a biodegradable polymer and applying the resulting melt onto the physiologically active agent layer 3 which has been formed on the surface of the stent body 2 as mentioned above. A method can include dissolving a biodegradable polymer in a solvent and dipping the stent body 2 which has the physiologically active agent layer 3 formed thereon in the resulting solution, followed by solvent removal by evaporation or the like. A method can include spraying the foregoing solution onto the stent body 2 which has the physiologically active agent layer 3 formed thereon, followed by solvent removal by evaporation or the like.

For example, the dipping and spraying methods which employ a solution dissolving only a biodegradable polymer in a solvent are simple and exemplary in the case where the biodegradable polymer can be dissolved in a solvent which makes the surface of the stent body 2 which has the physiologically active agent layer 3 formed thereon highly wettable.

The stent body 2 may have on the surface thereof the layer of biodegradable polymer 5 which contains the physiologically active agent 6 dispersed or embedded therein.

An exemplary stent may be constructed such that the stent body 2 has thereon a layer of biodegradable polymer containing a physiologically active agent dispersed or embedded therein, for example, a layer of mixture of a biodegradable polymer and a physiologically active agent. The mixing ratio (mass part) of the biodegradable polymer to the physiologically active agent can be from 99:1 to 1:99, for example, from 90:10 to 10:90, for example, from 70:30 to 30:70.

The layer of biodegradable polymer 5 containing the physiologically active agent 6 dispersed therein (or mixed therewith) can have an adequate thickness not harmful to the performance of the stent body 2, such as, for example, easy delivery to the lesion and low stimulus to the blood vessel. An exemplary thickness can be 1 to 100 μm, for example, 1 to 50 μm, for example, 1 to 20 μm, for example, such that the physiologically active agent fully produces its effect.

Any suitable method may be employed without specific restrictions to form the layer of biodegradable polymer 5 containing the physiologically active agent 6 dispersed or embedded therein on the surface of the stent body 2. It is exemplified below. A method can include melting a biodegradable polymer and a physiologically active agent and applying the resulting melt onto the surface of the stent body 2. A method can include dissolving a biodegradable polymer and a physiologically active agent in a solvent and dipping the stent body 2 in the resulting solution, followed by solvent removal by evaporation or the like. A method can include spraying the foregoing solution onto the stent body 2, followed by solvent removal by evaporation or the like.

For example, the dipping and spraying methods which employ a solution dissolving a biodegradable polymer and a physiologically active agent in a solvent are simple and exemplary in the case where the biodegradable polymer and the physiologically active agent can be dissolved in a solvent which makes the surface of the stent body 2 highly wettable.

In an exemplary embodiment, the layer that releases a physiologically active agent can be formed from a composition containing a physiologically active agent and a biodegradable polymer, and it can be the physiologically active agent layer 3, the biodegradable polymer layer 4, or the layer of biodegradable polymer 5 containing the physiologically active agent 6 dispersed therein. The layer that releases a physiologically active agent does not need to cover the entire surface of the linear members constituting the stent body. The layer that releases a physiologically active agent can cover at least a portion of the surface of the linear elements C constituting the stent body.

The layer that releases a physiologically active agent can cover 1 to 100%, for example, 50 to 100%, of the entire surface area of the stent body.

The stent body 2 may contain the physiologically active agent 6 dispersed or embedded therein. The stent body 2 of such structure may be produced by any suitable method. A desirable simple method can include incorporating a physiologically active agent into the bioabsorbable aliphatic polyester and the aromatic compound at the time of melt molding. Another method can include mixing a physiologically active agent with a mixture of the bioabsorbable aliphatic polyester and the aromatic compound. The resulting mixture can be biodegradable as well as capable of releasing the biological physiologically active agent. Thus the biological physiologically active agent can suppress inflammation that results from biodegradation of the stent body.

The physiologically active agent 3 can dissolve and diffuse into the biodegradable polymer layer 4 or the biodegradable polymer layer 4 or 5 decomposes in the living body, so that, for example, the physiologically active agent 3 or 6 is entirely released in the living body. At the same time, the stent body 2 decomposes in the living body. In this way, for example, all the constituents of the stent can disappear eventually.

The stent 1 can be expanded in any suitable way without specific restrictions. In case of self-expandable type, it can expand in its radial direction by its own restoring force when it is released from the force that keeps the stent folded up small. In the case of balloon-expandable type, the stent can be expanded in its radial direction by external force applied from the inside of the stent.

### EXAMPLES

In order to demonstrate its effect, exemplary aspects will be described in more detail with reference to the following Examples and Comparative Examples, which are not intended to restrict the technical scope thereof.

### Example 1

A mixture was prepared by dry blending from 100 pbw of polylactic acid (having a weight-average molecular weight of 123,000, made by DURECT Corporation) and 1 pbw of mandelic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 90° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm, by using an autograph (Model AG-IS, made by Shimadzu Corporation). It was found that the radial force (rebound) was 139 gf.

The same procedure as mentioned above was repeated to produce the stent of the structure as shown in FIG. 1. It was found that this stent has a radial force (rebound) of 164 gf.

### Example 2

A mixture was prepared by dry blending from 100 pbw of polylactic acid (made by DURECT Corporation, the same one as used in Example 1) and 1 pbw of 4-hydroxycinnamic acid (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 80° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 131 gf.

The same procedure as mentioned above was repeated to produce the stent of the structure as shown in FIG. 1. It was found that this stent has a radial force (rebound) of 155 gf.

### Example 3

A mixture was prepared by dry blending from 100 pbw of polylactic acid (made by DURECT Corporation, the same one as used in Example 1) and 1 pbw of 4-hydroxybenzoic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 85° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 150 gf.

The same procedure as mentioned above was repeated to produce the stent of the structure as shown in FIG. 1. It was found that this stent has a radial force (rebound) of 173 gf.

### Example 4

A mixture was prepared by dry blending from 100 pbw of polylactic acid (made by DURECT Corporation, the same one as used in Example 1) and 1 pbw of 3-iodo-L-tyrosine (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 90° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 159 gf. This stent was visible under X-ray radiography.

The same procedure as mentioned above was repeated to produce the stent of the structure as shown in FIG. 1. It was found that this stent has a radial force (rebound) of 183 gf. This stent was visible under X-ray radiography.

### Example 5

A mixture was prepared by dry blending from 100 pbw of polylactic acid (made by DURECT Corporation, the same one as used in Example 1) and 1 pbw of 3,5-diiodo-salicylic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 90° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 150 gf. This stent was visible under X-ray radiography.

The same procedure as mentioned above was repeated to produce the stent of the structure as shown in FIG. 1. It was found that this stent has a radial force (rebound) of 163 gf. This stent was visible under X-ray radiography.

### Example 6

A mixture was prepared by dry blending from 100 pbw of polylactic acid (made by DURECT Corporation, the same one as used in Example 1) and 7 pbw of mandelic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 90° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 139 gf.

The same procedure as mentioned above was repeated to produce the stent of the structure as shown in FIG. 1. It was found that this stent has a radial force (rebound) of 164 gf.

### Example 7

A mixture was prepared by dry blending from 100 pbw of polylactic acid (made by DURECT Corporation, the same one as used in Example 1) and 7 pbw of 4-hydroxycinnamic acid (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 80° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 131 gf.

The same procedure as mentioned above was repeated to produce the stent of the structure as shown in FIG. 1. It was found that this stent has a radial force (rebound) of 155 gf.

### Example 8

A mixture was prepared by dry blending from 100 pbw of polylactic acid (made by DURECT Corporation, the same one as used in Example 1) and 7 pbw of 4-hydroxybenzoic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 85° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 150 gf.

The same procedure as mentioned above was repeated to produce the stent of the structure as shown in FIG. 1. It was found that this stent has a radial force (rebound) of 173 gf.

### Example 9

A mixture was prepared by dry blending from 100 pbw of polylactic acid (made by DURECT Corporation, the same one as used in Example 1) and 7 pbw of 3-iodo-L-tyrosine (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 90° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 159 gf. This stent was visible under X-ray radiography.

The same procedure as mentioned above was repeated to produce the stent of the structure as shown in FIG. 1. It was found that this stent has a radial force (rebound) of 183 gf. This stent was visible under X-ray radiography.

### Example 10

A mixture was prepared by dry blending from 100 pbw of polylactic acid (made by DURECT Corporation, the same one as used in Example 1) and 7 pbw of 3,5-diiodo-salicylic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 90° C. and 0.5 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 150 gf. This stent was visible under X-ray radiography.

The same procedure as mentioned above was repeated to produce the stent of the structure as shown in FIG. 1. It was found that this stent has a radial force (rebound) of 163 gf. This stent was visible under X-ray radiography.

### Example 11

A mixture was prepared by dry blending from 100 pbw of lactic acid-trimethylene carbonate copolymer having a weight-average molecular weight of 100,000 (made by Taki Chemical Co., Ltd.) and 1 pbw of mandelic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 60° C. and 1.0 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 131 gf.

The same procedure as mentioned above was repeated to produce the stent of the structure as shown in FIG. 1. It was found that this stent has a radial force (rebound) of 158 gf.

### Example 12

A mixture was prepared by dry blending from 100 pbw of lactic acid-trimethylene carbonate copolymer (made by Taki Chemical Co., Ltd., the same one as used in Example 11) and 1 pbw of 4-hydroxycinnamic acid (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 75° C. and 0.8 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 123 gf.

The same procedure as mentioned above was repeated to produce the stent of the structure as shown in FIG. 1. It was found that this stent has a radial force (rebound) of 143 gf.

### Example 13

A mixture was prepared by dry blending from 100 pbw of lactic acid-trimethylene carbonate copolymer (made by Taki Chemical Co., Ltd., the same one as used in Example 11) and 1 pbw of 4-hydroxybenzoic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 55° C. and 0.9 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 140 gf.

The same procedure as mentioned above was repeated to produce the stent of the structure as shown in FIG. 1. It was found that this stent has a radial force (rebound) of 153 gf.

### Example 14

A mixture was prepared by dry blending from 100 pbw of lactic acid-trimethylene carbonate copolymer (made by Taki Chemical Co., Ltd., the same one as used in Example 11) and 1 pbw of 3-iodo-L-tyrosine (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 65° C. and 1.2 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 139 gf. This stent was visible under X-ray radiography.

The same procedure as mentioned above was repeated to produce the stent of the structure as shown in FIG. 1. It was found that this stent has a radial force (rebound) of 146 gf. This stent was visible under X-ray radiography.

### Example 15

A mixture was prepared by dry blending from 100 pbw of lactic acid-trimethylene carbonate copolymer (made by Taki Chemical Co., Ltd., the same one as used in Example 11) and 1 pbw of 3,5-diiodo-salicylic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 60° C. and 0.9 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 136 gf. This stent was visible under X-ray radiography.

The same procedure as mentioned above was repeated to produce the stent of the structure as shown in FIG. 1. It was found that this stent has a radial force (rebound) of 146 gf. This stent was visible under X-ray radiography.

### Example 16

A mixture was prepared by dry blending from 100 pbw of lactic acid-trimethylene carbonate copolymer (made by Taki Chemical Co., Ltd., the same one as used in Example 11) and 7 pbw of mandelic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 60° C. and 1.0 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 123 gf.

The same procedure as mentioned above was repeated to produce the stent of the structure as shown in FIG. 1. It was found that this stent has a radial force (rebound) of 141 gf.

### Example 17

A mixture was prepared by dry blending from 100 pbw of lactic acid-trimethylene carbonate copolymer (made by Taki Chemical Co., Ltd., the same one as used in Example 11) and 7 pbw of 4-hydroxycinnamic acid (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 75° C. and 0.8 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 120 gf.

The same procedure as mentioned above was repeated to produce the stent of the structure as shown in FIG. 1. It was found that this stent has a radial force (rebound) of 134 gf.

### Example 18

A mixture was prepared by dry blending from 100 pbw of lactic acid-trimethylene carbonate copolymer (made by Taki Chemical Co., Ltd., the same one as used in Example 11) and 7 pbw of 4-hydroxybenzoic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 55° C. and 0.9 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 136 gf.

The same procedure as mentioned above was repeated to produce the stent of the structure as shown in FIG. 1. It was found that this stent has a radial force (rebound) of 156 gf.

### Example 19

A mixture was prepared by dry blending from 100 pbw of lactic acid-trimethylene carbonate copolymer (made by Taki Chemical Co., Ltd., the same one as used in Example 11) and 7 pbw of 3-iodo-L-tyrosine (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 65° C. and 1.2 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 146 gf. This stent was visible under X-ray radiography.

The same procedure as mentioned above was repeated to produce the stent of the structure as shown in FIG. 1. It was found that this stent has a radial force (rebound) of 159 gf. This stent was visible under X-ray radiography.

### Example 20

A mixture was prepared by dry blending from 100 pbw of lactic acid-trimethylene carbonate copolymer (made by Taki Chemical Co., Ltd., the same one as used in Example 11) and 7 pbw of 3,5-diiodo-salicylic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 60° C. and 0.9 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 141 gf. This stent was visible under X-ray radiography.

The same procedure as mentioned above was repeated to produce the stent of the structure as shown in FIG. 1. It was found that this stent has a radial force (rebound) of 156 gf. This stent was visible under X-ray radiography.

### Example 21

A mixture was prepared by dry blending from 100 pbw of polyglycolic acid having a weight-average molecular weight of 159,800 (made by DURECT Corporation) and 1 pbw of mandelic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 145 gf.

The same procedure as mentioned above was repeated to produce the stent of the structure as shown in FIG. 1. It was found that this stent has a radial force (rebound) of 173 gf.

### Example 22

A mixture was prepared by dry blending from 100 pbw of polyglycolic acid (made by DURECT Corporation, the same one as used in Example 21) and 1 pbw of 4-hydroxycinnamic acid (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 138 gf.

The same procedure as mentioned above was repeated to produce the stent of the structure as shown in FIG. 1. It was found that this stent has a radial force (rebound) of 162 gf.

### Example 23

A mixture was prepared by dry blending from 100 pbw of polyglycolic acid (made by DURECT Corporation, the same one as used in Example 21) and 1 pbw of 4-hydroxybenzoic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 160 gf.

The same procedure as mentioned above was repeated to produce the stent of the structure as shown in FIG. 1. It was found that this stent has a radial force (rebound) of 186 gf.

### Example 24

A mixture was prepared by dry blending from 100 pbw of polyglycolic acid (made by DURECT Corporation, the same one as used in Example 21) and 1 pbw of 3-iodo-L-tyrosine (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 171 gf. This stent was visible under X-ray radiography.

The same procedure as mentioned above was repeated to produce the stent of the structure as shown in FIG. 1. It was found that this stent has a radial force (rebound) of 196 gf. This stent was visible under X-ray radiography.

### Example 25

A mixture was prepared by dry blending from 100 pbw of polyglycolic acid (made by DURECT Corporation, the same one as used in Example 21) and 1 pbw of 3,5-diiodo-salicylic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 163 gf. This stent was visible under X-ray radiography.

The same procedure as mentioned above was repeated to produce the stent of the structure as shown in FIG. 1. It was found that this stent has a radial force (rebound) of 178 gf. This stent was visible under X-ray radiography.

### Example 26

A mixture was prepared by dry blending from 100 pbw of polyglycolic acid (made by DURECT Corporation, the same one as used in Example 21) and 7 pbw of mandelic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 149 gf.

The same procedure as mentioned above was repeated to produce the stent of the structure as shown in FIG. 1. It was found that this stent has a radial force (rebound) of 171 gf.

### Example 27

A mixture was prepared by dry blending from 100 pbw of polyglycolic acid (made by DURECT Corporation, the same one as used in Example 21) and 7 pbw of 4-hydroxycinnamic acid (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 146 gf.

The same procedure as mentioned above was repeated to produce the stent of the structure as shown in FIG. 1. It was found that this stent has a radial force (rebound) of 170 gf.

### Example 28

A mixture was prepared by dry blending from 100 pbw of polyglycolic acid (made by DURECT Corporation, the same one as used in Example 21) and 7 pbw of 4-hydroxybenzoic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 155 gf.

The same procedure as mentioned above was repeated to produce the stent of the structure as shown in FIG. 1. It was found that this stent has a radial force (rebound) of 184 gf.

### Example 29

A mixture was prepared by dry blending from 100 pbw of polyglycolic acid (made by DURECT Corporation, the same one as used in Example 21) and 7 pbw of 3-iodo-L-tyrosine (made by Tokyo Chemical Industry Co., Ltd.). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 167 gf. This stent was visible under X-ray radiography.

The same procedure as mentioned above was repeated to produce the stent of the structure as shown in FIG. 1. It was found that this stent has a radial force (rebound) of 186 gf. This stent was visible under X-ray radiography.

### Example 30

A mixture was prepared by dry blending from 100 pbw of polyglycolic acid (made by DURECT Corporation, the same one as used in Example 21) and 7 pbw of 3,5-diiodo-salicylic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm by means of an autograph (Model AG-IS, made by Shimadzu Seisakusho). It was found that the radial force (rebound) was 163 gf. This stent was visible under X-ray radiography.

The same procedure as mentioned above was repeated to produce the stent of the structure as shown in FIG. 1. It was found that this stent has a radial force (rebound) of 177 gf. This stent was visible under X-ray radiography.

### Comparative Example 1

Polylactic acid (made by DURECT Corporation, the same one as used in Example 1) was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 90° C. and 0.3 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 92 gf.

The same procedure as mentioned above was repeated to produce the stent of the structure as shown in FIG. 1. It was found that this stent has a radial force (rebound) of 96 gf.

### Comparative Example 2

Lactic acid-trimethylene carbonate copolymer (made by Taki Chemical Co., Ltd., the same one as used in Example 11) was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 60° C. and 1.0 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 83 gf.

The same procedure as mentioned above was repeated to produce the stent of the structure as shown in FIG. 1. It was found that this stent has a radial force (rebound) of 89 gf.

### Comparative Example 3

Polyglycolic acid (made by DURECT Corporation, the same one as used in Example 21) was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 45° C. and 2.0 MPa for 300 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 89 gf.

The same procedure as mentioned above was repeated to produce the stent of the structure as shown in FIG. 1. It was found that this stent has a radial force (rebound) of 93 gf.

### Comparative Example 4

Polycaprolactone having a weight-average molecular weight of 115,000 (made by DURECT Corporation) was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 40° C. and 0.1 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 38 gf.

The same procedure as mentioned above was repeated to produce the stent of the structure as shown in FIG. 1. It was found that this stent has a radial force (rebound) of 42 gf.

### Comparative Example 5

A mixture was prepared by dry blending from 100 pbw of polylactic acid (made by DURECT Corporation, the same one as used in Example 1) and 1 pbw of succinic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 80° C. and 0.4 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 83 gf.

### Comparative Example 6

A mixture was prepared by dry blending from 100 pbw of polylactic acid (made by DURECT Corporation, the same one as used in Example 1) and 1 pbw of adipic acid (made by Sigma-Aldrich Corporation). The resulting mixture was formed into a tube, measuring 1.38 mm in outside diameter and 0.45 mm in inside diameter, by extrusion molding (with Labo Plastomill made by Toyo Seiki Seisaku-Sho, Ltd.). The resulting tube underwent blow molding by introduction of pressurized dry nitrogen at 80° C. and 0.4 MPa for 120 seconds. Thus there was obtained a blown tube measuring 3.00 mm in outside diameter and 2.70 mm in inside diameter. The thus obtained blown tube was fabricated by means of ArF excimer laser (193 nm) to give the stent of the structure as shown in FIG. 8.

The stent of 3.00 mm in outside diameter and cut to a length of 10 mm was tested for radial force (rebound) exerted by it after compression by 1 mm in the same way as in Example 1. It was found that the radial force (rebound) was 86 gf.

### Comparative Example 7

A stent of stainless steel (SUS 316L) was prepared which has the structure as shown in FIG. 1 and measures 3.00 mm in outside diameter and 10 mm in length. This stent was tested for radial force (rebound) exerted by it after compression by 1 mm in the same way as in Example 1. It was found that the radial force (rebound) was 196 gf.

Tables 1 to 3 below summarize the results of Examples 1 to 10 and Comparative Examples 1 to 7, the results of Examples 11 to 20 and Comparative Examples 1 to 7, and the results of Examples 21 to 30 and Comparative Examples 1 to 7, respectively.

TABLE 1

| | | | Aromatic compound | | Radial force of | Radial force of |
|---|---|---|---|---|---|---|
| | Bioabsorbable aliphatic polyester | | | Mixing ratio *¹ | stent of FIG. 8 | stent of FIG. 1 |
| | Name | Mw | Name | (by mass) | (gf) | (gf) |
| Example 1 | Polylactic acid | 123,000 | Mandelic acid | 1 | 139 | 164 |
| Example 2 | Polylactic acid | 123,000 | 4-hydroxycinnamic acid | 1 | 131 | 155 |
| Example 3 | Polylactic acid | 123,000 | 4-hydroxybenzoic acid | 1 | 150 | 173 |
| Example 4 | Polylactic acid | 123,000 | 3-iodo-L-tyrosine | 1 | 159 | 183 |
| Example 5 | Polylactic acid | 123,000 | 3,5-diiodo-salicylic acid | 1 | 150 | 163 |
| Example 6 | Polylactic acid | 123,000 | Mandelic acid | 7 | 139 | 164 |
| Example 7 | Polylactic acid | 123,000 | 4-hydroxycinnamic acid | 7 | 131 | 155 |
| Example 8 | Polylactic acid | 123,000 | 4-hydroxybenzoic acid | 7 | 150 | 173 |
| Example 9 | Polylactic acid | 123,000 | 3-iodo-L-tyrosine | 7 | 159 | 183 |
| Example 10 | Polylactic acid | 123,000 | 3,5-diiodo-salicylic acid | 7 | 150 | 163 |
| Comp. Ex. 1 | Polylactic acid | 123,000 | — | — | 92 | 96 |
| Comp. Ex. 2 | Lactic acid-trimethylene | 100,000 | — | — | 83 | 89 |
| | carbonate copolymer | | | | | |
| Comp. Ex. 3 | Polyglycolic acid | 159,800 | — | — | 89 | 93 |
| Comp. Ex. 4 | Polycaprolactone | 115,000 | — | — | 38 | 42 |
| Comp. Ex. 5 | Polylactic acid | 123,000 | Scuccinic acid *² | 1 | 83 | N.D. |
| Comp. Ex. 6 | Polylactic acid | 123,000 | Adipic acid *² | 1 | 86 | N.D. |

| | | | |
|---|---|---|---|
| Comp. Ex. 7 | Stent of stainless steel | N.D. | 196 |

Mw = Weight-average molecular weight, N.D. = not determined, Comp. Ex. = Comparative Example
*¹ Mixing ratio (by mass) of aromatic compound to 100 pbw of bioabsorbable aliphatic polyester
*² Succinic acid and adipic acid are aliphatic compounds.

TABLE 2

| | | | Aromatic compound | | Radial force of | Radial force of |
|---|---|---|---|---|---|---|
| | Bioabsorbable aliphatic polyester | | | Mixing ratio *¹ | stent of FIG. 8 | stent of FIG. 1 |
| | Name | Mw | Name | (by mass) | (gf) | (gf) |
| Example 11 | Lactic acid-trimethylene | 100,000 | Mandelic acid | 1 | 131 | 158 |
| | carbonate copolymer | | | | | |
| Example 12 | Lactic acid-trimethylene | 100,000 | 4-hydroxycinnamic acid | 1 | 123 | 143 |
| | carbonate copolymer | | | | | |
| Example 13 | Lactic acid-trimethylene | 100,000 | 4-hydroxybenzoic acid | 1 | 140 | 153 |
| | carbonate copolymer | | | | | |
| Example 14 | Lactic acid-trimethylene | 100,000 | 3-iodo-L-tyrosine | 1 | 139 | 146 |
| | carbonate copolymer | | | | | |
| Example 15 | Lactic acid-trimethylene | 100,000 | 3,5-diiodo-salicylic acid | 1 | 136 | 146 |
| | carbonate copolymer | | | | | |
| Example 16 | Lactic acid-trimethylene | 100,000 | Mandelic acid | 7 | 123 | 141 |
| | carbonate copolymer | | | | | |
| Example 17 | Lactic acid-trimethylene | 100,000 | 4-hydroxycinnamic acid | 7 | 120 | 134 |
| | carbonate copolymer | | | | | |
| Example 18 | Lactic acid-trimethylene | 100,000 | 4-hydroxybenzoic acid | 7 | 136 | 156 |
| | carbonate copolymer | | | | | |
| Example 19 | Lactic acid-trimethylene | 100,000 | 3-iodo-L-tyrosine | 7 | 146 | 159 |
| | carbonate copolymer | | | | | |
| Example 20 | Lactic acid-trimethylene | 100,000 | 3,5-diiodo-salicylic acid | 7 | 141 | 156 |
| | carbonate copolymer | | | | | |
| Comp. Ex. 1 | Polylactic acid | 123,000 | — | — | 92 | 96 |
| Comp. Ex. 2 | Lactic acid-trimethylene | 100,000 | — | — | 83 | 89 |
| | carbonate copolymer | | | | | |
| Comp. Ex. 3 | Polyglycolic acid | 159,800 | — | — | 89 | 93 |
| Comp. Ex. 4 | Polycaprolactone | 115,000 | — | — | 38 | 42 |
| Comp. Ex. 5 | Polylactic acid | 123,000 | Scuccinic acid *² | 1 | 83 | N.D. |
| Comp. Ex. 6 | Polylactic acid | 123,000 | Adipic acid *² | 1 | 86 | N.D. |

| | | | |
|---|---|---|---|
| Comp. Ex. 7 | Stent of stainless steel | N.D. | 196 |

Mw = Weight-average molecular weight, N.D. = not determined, Comp. Ex. = Comparative Example
*¹ Mixing ratio (by mass) of aromatic compound to 100 pbw of bioabsorbable aliphatic polyester
*² Succinic acid and adipic acid are aliphatic compounds.

TABLE 3

| | | | Aromatic compound | | Radial force of | Radial force of |
|---|---|---|---|---|---|---|
| | Bioabsorbable aliphatic polyester | | | Mixing ratio *¹ | stent of FIG. 8 | stent of FIG. 1 |
| | Name | Mw | Name | (by mass) | (gf) | (gf) |
| Example 21 | Polyglycolic acid | 159,800 | Mandelic acid | 1 | 145 | 173 |
| Example 22 | Polyglycolic acid | 159,800 | 4-hydroxycinnamic acid | 1 | 138 | 162 |
| Example 23 | Polyglycolic acid | 159,800 | 4-hydroxybenzoic acid | 1 | 160 | 186 |
| Example 24 | Polyglycolic acid | 159,800 | 3-iodo-L-tyrosine | 1 | 171 | 196 |
| Example 25 | Polyglycolic acid | 159,800 | 3,5-diiodo-salicylic acid | 1 | 163 | 178 |
| Example 26 | Polyglycolic acid | 159,800 | Mandelic acid | 7 | 149 | 171 |
| Example 27 | Polyglycolic acid | 159,800 | 4-hydroxycinnamic acid | 7 | 146 | 170 |
| Example 28 | Polyglycolic acid | 159,800 | 4-hydroxybenzoic acid | 7 | 155 | 184 |
| Example 29 | Polyglycolic acid | 159,800 | 3-iodo-L-tyrosine | 7 | 167 | 186 |
| Example 30 | Polyglycolic acid | 159,800 | 3,5-diiodo-salicylic acid | 7 | 163 | 177 |
| Comp. Ex. 1 | Polylactic acid | 123,000 | — | — | 92 | 96 |
| Comp. Ex. 2 | Lactic acid-trimethylene | 100,000 | — | — | 83 | 89 |
| | carbonate copolymer | | | | | |
| Comp. Ex. 3 | Polyglycolic acid | 159,800 | — | — | 89 | 93 |
| Comp. Ex. 4 | Polycaprolactone | 115,000 | — | — | 38 | 42 |
| Comp. Ex. 5 | Polylactic acid | 123,000 | Scuccinic acid *² | 1 | 83 | N.D. |
| Comp. Ex. 6 | Polylactic acid | 123,000 | Adipic acid *² | 1 | 86 | N.D. |

| | | | |
|---|---|---|---|
| Comp. Ex. 7 | Stent of stainless steel | N.D. | 196 |

Mw = Weight-average molecular weight, N.D. = not determined, Comp. Ex. = Comparative Example
*¹ Mixing ratio (by mass) of aromatic compound to 100 pbw of bioabsorbable aliphatic polyester
*² Succinic acid and adipic acid are aliphatic compounds.

The detailed description above describes features and aspects of embodiments of a stent disclosed by way of example. The invention is not limited, however, to the precise embodiments and variations described. Changes, modifications and equivalents can be employed by one skilled in the art without departing from the spirit and scope of the invention as defined in the appended claims. It is expressly intended that all such changes, modifications and equivalents which fall within the scope of the claims are embraced by the claims.

What is claimed is:

1. A bioabsorbable stent formed from a mixture comprising a bioabsorbable aliphatic polyester and an aromatic compound having one or more aromatic rings.

2. The bioabsorbable stent as defined in claim 1, wherein the bioabsorbable aliphatic polyester and the aromatic compound are mixed in a ratio (by mass) ranging from 100:0.1 to 100:9.

3. The bioabsorbable stent as defined in claim 1, wherein the aromatic compound has a hydroxyl group or carboxyl group.

4. The bioabsorbable stent as defined in claim 1, wherein the aromatic compound includes at least one selected from the group consisting of 2-hydroxybenzoic acid, 3-hydroxybenzoic acid, 4-hydroxybenzoic acid, 2,3-dihydroxybenzoic acid, 2,4-dihydroxybenzoic acid, 2,5-dihydroxybenzoic acid, 2,6-dihydroxybenzoic acid, 3,5-dihydroxybenzoic acid, 2-hydroxycinnamic acid, 3-hydroxycinnamic acid, 4-hydroxycinnamic acid, 4-hydroxy-2-methoxycinnamic acid, 4-hydroxy-3-methoxycinnamic acid, 3,4-dihydroxycinnamic acid, mandelic acid, and tyrosine and iodide compounds thereof.

5. The bioabsorbable stent as defined in claim 1, wherein the bioabsorbable aliphatic polyester includes at least one selected from the group consisting of polylactic acid, polyglycolic acid, copolymer of lactic acid and glycolic acid, polycaprolactone, copolymer of lactic acid and caprolactone, copolymer of glycolic acid and caprolactone, polytrimethylene carbonate, copolymer of lactic acid and trimethylene carbonate, copolymer of glycolic acid and trimethylene carbonate, polydioxanone, polyethylene succinate, polybutylene succinate, and polybutylene succinate-adipate.

6. The bioabsorbable stent as defined in claim 1, which is formed by blow molding the mixture comprising the bioabsorbable aliphatic polyester and the aromatic compound having one or more aromatic rings.

7. The bioabsorbable stent as defined in claim 2, wherein the aromatic compound has a hydroxyl group or carboxyl group.

8. The bioabsorbable stent as defined in claim 2, wherein the aromatic compound includes at least one selected from the group consisting of 2-hydroxybenzoic acid, 3-hydroxybenzoic acid, 4-hydroxybenzoic acid, 2,3-dihydroxybenzoic acid, 2,4-dihydroxybenzoic acid, 2,5-dihydroxybenzoic acid, 2,6-dihydroxybenzoic acid, 3,5-dihydroxybenzoic acid, 2-hydroxycinnamic acid, 3-hydroxycinnamic acid, 4-hydroxycinnamic acid, 4-hydroxy-2-methoxycinnamic acid, 4-hydroxy-3-methoxycinnamic acid, 3,4-dihydroxycinnamic acid, mandelic acid, and tyrosine and iodide compounds thereof.

9. The bioabsorbable stent as defined in claim 3, wherein the aromatic compound includes at least one selected from the group consisting of 2-hydroxybenzoic acid, 3-hydroxybenzoic acid, 4-hydroxybenzoic acid, 2,3-dihydroxybenzoic acid, 2,4-dihydroxybenzoic acid, 2,5-dihydroxybenzoic acid, 2,6-dihydroxybenzoic acid, 3,5-dihydroxybenzoic acid, 2-hydroxycinnamic acid, 3-hydroxycinnamic acid, 4-hydroxycinnamic acid, 4-hydroxy-2-methoxycinnamic acid, 4-hydroxy-3-methoxycinnamic acid, 3,4-dihydroxycinnamic acid, mandelic acid, and tyrosine and iodide compounds thereof.

10. The bioabsorbable stent as defined in claim 2, wherein the bioabsorbable aliphatic polyester includes at least one selected from the group consisting of polylactic acid, polyglycolic acid, copolymer of lactic acid and glycolic acid, polycaprolactone, copolymer of lactic acid and caprolactone, copolymer of glycolic acid and caprolactone, polytrimethylene carbonate, copolymer of lactic acid and trimethylene carbonate, copolymer of glycolic acid and trimethylene carbonate, polydioxanone, polyethylene succinate, polybutylene succinate, and polybutylene succinate-adipate.

11. The bioabsorbable stent as defined in claim 3, wherein the bioabsorbable aliphatic polyester includes at least one selected from the group consisting of polylactic acid, polyglycolic acid, copolymer of lactic acid and glycolic acid, polycaprolactone, copolymer of lactic acid and caprolactone, copolymer of glycolic acid and caprolactone, polytrimethylene carbonate, copolymer of lactic acid and trimethylene carbonate, copolymer of glycolic acid and trimethylene carbonate, polydioxanone, polyethylene succinate, polybutylene succinate, and polybutylene succinate-adipate.

12. The bioabsorbable stent as defined in claim 4, wherein the bioabsorbable aliphatic polyester includes at least one selected from the group consisting of polylactic acid, polyglycolic acid, copolymer of lactic acid and glycolic acid, polycaprolactone, copolymer of lactic acid and caprolactone, copolymer of glycolic acid and caprolactone, polytrimethylene carbonate, copolymer of lactic acid and trimethylene carbonate, copolymer of glycolic acid and trimethylene carbonate, polydioxanone, polyethylene succinate, polybutylene succinate, and polybutylene succinate-adipate.

13. The bioabsorbable stent as defined in claim 2, which is formed by blow molding the mixture comprising the bioabsorbable aliphatic polyester and the aromatic compound having one or more aromatic rings.

14. The bioabsorbable stent as defined in claim 3, which is formed by blow molding the mixture comprising the bioabsorbable aliphatic polyester and the aromatic compound having one or more aromatic rings.

15. The bioabsorbable stent as defined in claim 4, which is formed by blow molding the mixture comprising the bioabsorbable aliphatic polyester and the aromatic compound having one or more aromatic rings.

16. The bioabsorbable stent as defined in claim 5, which is formed by blow molding the mixture comprising the bioabsorbable aliphatic polyester and the aromatic compound having one or more aromatic rings.

17. The bioabsorbable stent as defined in claim 1, wherein the bioabsorbable aliphatic polyester has a weight-average molecular weight of 10,000 to 3,000,000.

18. The bioabsorbable stent as defined in claim 3, wherein the bioabsorbable aliphatic polyester has a reactive functional group, and wherein the hydroxyl or carboxyl group of the aromatic compound forms a chemical linkage with the reactive functional group of the bioabsorbable aliphatic polyester.

19. The bioabsorbable stent as defined in claim 1, wherein the one or more aromatic rings of the aromatic compound causes molecular chains of the bioabsorbable aliphatic polyester to regularly arrange by a stacking action of the one or more aromatic rings.

20. A method of forming the bioabsorbable stent as defined in claim 1, the method comprising forming the bioabsorbable stent by subjecting the mixture comprising the bioabsorbable aliphatic polyester and the aromatic compound having one or more aromatic rings to blow molding.
